(19) United States
(12) Patent Application Publication
(10) Pub. No.: US 2010/0125197 A1
(43) Pub. Date: May 20, 2010

(54) METHOD AND APPARATUS FOR ADDRESSING VASCULAR STENOTIC LESIONS

(76) Inventor: Robert S. Fishel, Delray Beach, FL (US)

Correspondence Address: MCHALE & SLAVIN, P.A., 2855 PGA BLVD, PALM BEACH GARDENS, FL, 33410, US

(21) Appl. No.: 12/476,429

(22) Filed: Jun. 2, 2009

Related U.S. Application Data
(60) Provisional application No. 61/115,769, filed on Nov. 18, 2008.
(60) Provisional application No. 61/116,964, filed on Nov. 21, 2008.

(51) Int. Cl. A61B 8/04; A61B 5/00

(52) U.S. Cl. 600/437; 600/300

(57) ABSTRACT

A method and apparatus are provided for addressing vascular stenotic lesions. The geometry of a stenotic lesion zone is determined and is used to determine pressure drop of blood flow across the lesion zone or a portion thereof. The pressure drop value is used to determine a treatment regimen for the stenotic lesion zone. The geometry if preferably a three dimensional geometry. The apparatus provides pressure drop values to a physician so the physician can treat the patient if treatment is needed and provides direction for treatment options as to type and degree.

### RELATED APPLICATIONS

This application claims the benefit under 35 U.S.C. 119 (e) of U.S. Provisional Patent Application No. 61/115,769, filed Nov. 18, 2008, entitled, “Method For Modeling Of Hydrodynamic Pressure Drop Across A Series Of Coronary And Other Vascular Stenotic Lesion Sets Using Data Sets Acquired Via 3-Dimensional Intravascular Ultrasound”, and U.S. Provisional Patent Application No. 61/116,964, filed Nov. 21, 2008, entitled, “Method For Modeling Of Hydrodynamic Pressure Drop Across A Series Of Coronary And Other Vascular Stenotic Lesion Sets Using Data Sets Acquired Via 3-Dimensional Intravascular Ultrasound”, the entirety of which is incorporated herein by reference.

### FIELD OF INVENTION

The present invention relates to a method of addressing occlusions in blood vessels to determine a treatment method or strategy. The significance of vascular stenosis is determined by the depth, the shape and the length of the stentic lesion and is correlated to predetermined pressure drop values across the lesion set. The pressure drop value across stenotic area is used to assist in determining the potential treatments.

### BACKGROUND OF THE INVENTION

Coronary and other vascular stenotic lesions can cause medical problems for people. The extent of the disease can be severe and can result in heart attack (myocardial infarction) and stroke. Current methods of addressing stenosis suggest a “significant” stenosis of an arterial branch blood vessel (vein or artery) is one in which the stenotic lesion exceeds greater than 70% of the vessel's normal internal diameter (about 50% on an area basis). This method is conducted using fluoroscopy and a doctor estimating the dimensional change. Such a method however is overly simplistic and does not account for the fact that the clinically relevant measure of a significant stenosis is more accurately determined by the perfusion pressure drop across a stenotic zone of a blood vessel. Moreover, stenotic vascular lesions are usually not concentric or isolated in their extent but rather are eccentric elongated complex structures which can at times be found in tandem and/or not uniform along their length. A long series of 40-60% stenosis areas in a blood vessel may ultimately produce a distal perfusion pressure drop greater than an isolated 70% or greater axially short stenosis area. Currently, the only way to accurately determine this pressure drop across a lesion zone is to directly measure the differences in pressure by physically placing a pressure sensor proximal and distal to a vascular stenotic lesion zone. However, this pressure measurement means more invasive steps must be taken.

One method utilized which improves upon visual estimation of stenosis is through the use of intravascular ultrasound to obtain measurements of the internal dimensions of a vessel including the extent of stenotic burden in relation to the normal vascular endothelial wall. Recent advances in electroanatomical mapping have allowed for a magnetic phase sensor to be positioned along with the intravascular ultrasound imaging sensor thus allowing for three dimensional reconstruction of a blood vessel's stenotic segments along the entire course of the respective vascular branch. Thus, an entire blood vessel's course including eccentric stenotic segments all in three dimensions can now be imaged with electroanatomically guided intravascular ultrasound.

Pressure in a fluid flowing in a pipe or other contained space is known to drop in a predictable manner depending on the particular architecture of that pipe or bounded space and also on the viscosity of the fluid. For example, frictional pressure losses in fire hoses are calculated by firefighters to determine the pressure and thus reach of the water as it exits the nozzle and thus to determine if larger hoses or additional pressure pumps are needed to allow the water to reach the target fire.

### SUMMARY

The present invention involves the provision of a method of addressing vascular stenosis. The method includes the determining of the degree of stenosis along an occluded zone in a blood vessel by both length and transverse area of the zone. That information is then used to estimate a pressure drop across the entire occluded zone and individual portions of the zone if desired. The need for vascular treatment can then be determined and the locus and extent of treatment in the zone can also be determined based upon predetermined values of pressure drop for given parameters. The parameters can include blood viscosity, blood flow rate, blood flow rate range, extent and type of occlusion.

The present invention also involves the provision of an apparatus for addressing vascular stenosis. The apparatus includes stored information that correlates occlusion physical parameters to pressure drop of blood flow through an occlusion. The apparatus includes a measured data input and a means to output results of analysis of the data input. Recommended treatments may also be provided in accordance with desired outcomes.

### BRIEF DESCRIPTION OF THE DRAWINGS

FIG. 1 is side sectional view of a blood vessel showing a stenosis zone having tandem occlusion areas of stenotic lesions.

FIG. 2 is a schematic representation of an apparatus useful in addressing vascular stenotic lesions.

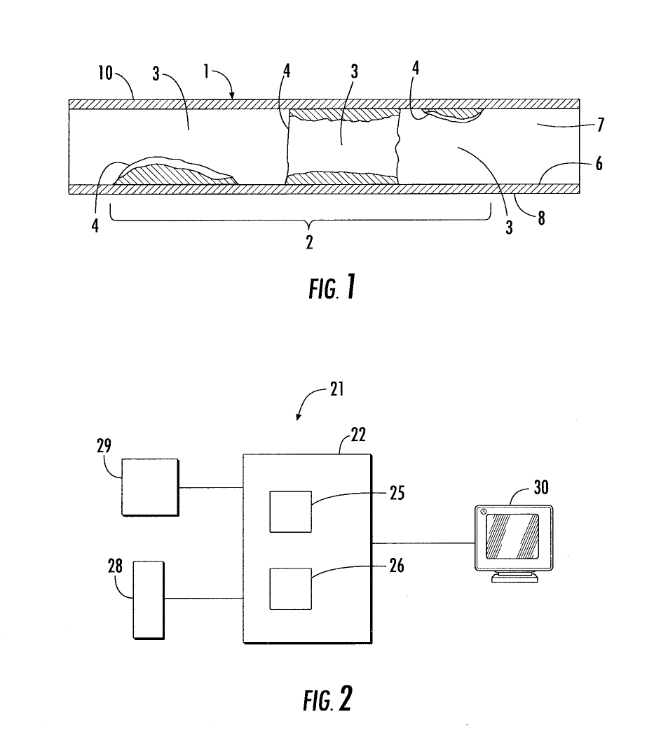

Like numbers used throughout this application represent like or similar parts and/or construction.

### DETAILED DESCRIPTION OF THE INVENTION

While the present invention is susceptible of embodiment in various forms, there is shown in the drawings and will hereinafter be described presently preferred embodiments with the understanding that the present disclosure is to be considered an exemplification of the invention and is not intended to limit the invention to the specific embodiments illustrated.

The reference numeral 1 designates generally a portion of a blood vessel have a stenosis zone 2 with a plurality of axially distributed occluded areas 3 of stenotic lesions 4. The lesions 5 extend inwardly from the epithelial wall 6 occluding the flow passage 7 between a proximal end portion 8 to a distal end portion 10. The blood flows in a direction from the proximal end 8 to the distal end 10.

The method includes addressing vascular stenotic lesions 3 in the vascular system of a patient like a human. The degree of stenosis is determined by measuring the size of the blood flow passage 7 at opposite ends of the stenosis zone 2 to provide a reference size for comparison as a norm. It is noted that different blood vessels have different sizes both as to other blood vessels and as to along their own length. These sizes are then a standard to compare the occluded zone 2 to. The stenosis zone is then examined to determine the degree of stenosis in the zone 3. This may be done using a three dimensional intravascular ultrasound imaging as is known in the art. Any suitable method that gives the open cross sectional over a desired length may be used. This will provide the degree of stenosis both by cross sectional area and by axial length or more accurately the size of the open passage along the stenosis zone 2 of interest. The zone of interest may have only one stenosis are 4 or the there may be tandem areas 4 in the zone 2. Once the stenosis zone 2 has been defined by size, a blood sample may be analyzed to determine its viscosity if desired. Other patient data may be gathered such as body mass, lifestyle (e.g., athletic activities), blood pressures, ejection fraction etc.

The pressure drop of the blood flowing through a stenosis zone 2 is then estimated for the conditions of interest, sedentary life, athletic activity needs, age, etc. The pressure drop is estimated using data generated from an algorithm. The algorithm may utilize theoretical flow equations that can be verified and/or adjusted experimentally. Portion of the algorithm may be derived statistically. Finite element analysis may also be used. The pressure drop estimation can be done using the algorithm for a calculation from input data including the geometry of the stenosis zone 2 and the occluded area 3 each time pressure drop values are needed. The algorithm may also be used to generate a look up table of pressure drop values and the pressure drop value(s) may be selected from the table for a set of input data values. The stenosis zone 2 may have the pressure drop calculated across the entire zone 2 and preferably for each of the individual occluded areas 3 in the zone 2. If there are multiple blood vessels or blood vessel branches involved, they may be evaluated separately and their impact on one another may also be determined. The effect of pulse flow and the change in flow over short time periods may be included for greater accuracy if desired. Once the pressure drops are determined and desired pressure at the distal end of a stenosis zone 2 is determined, a treatment regimen may be addressed. The total blood flow need will determine the tolerable pressure drop for a patient's needs. The degree and type of treatment may then be determined to achieve the desired pressure drop decrease which can be estimated by analyzing the decrease in pressure drop by evaluating the new pressure drop for each area as that area is enlarged with a stent or angioplasty or other treatment to enlarge the flow path at each area 3. Whether treatment is done at the distal end 10, the proximal end 8 or in between may be determined by the treatment's effect on the resulting pressure at the distal end 8. The change in pressure drop may also be determined by the algorithm to determine if the proposed treatment will effect sufficient change in pressure drop and hence blood flow.

The present invention also involves an apparatus usable in the process described above. The apparatus is designated generally as 21 and includes a data processing apparatus 22 such as a digital computer having a processor 25 and a memory 26. It has a data input means 28 such as a keyboard and/or a single or multiple channel data gathering device that is operable to input data representing the geometry of a stenosis zone. Preferably the geometry is three dimensional geometry. This can be done with an intravascular ultrasound or other device as desired. Such devices are well known in the art. Input data represents the transverse geometry or areas of a stenosis zone 2 along its length and will define the flow passage 7 contours. It can also provide data representative of the blood vessel of interest outside of the stenosis zone 2 to determine the degree of constriction in the zone 2. The data processing apparatus 22 is programmed to provide output from a pressure drop algorithm. The programming may include the algorithm and/or data generated by the algorithm which with the received input data of the stenosis zone geometry can provide pressure drop data for the zone 2 and/or the occluded areas 3. Generated data may be stored in a look up table or the like. The output data is provided on an output device 30 such as a monitor connected to the apparatus 22. The output values are determined by input values including at least one of stenosis zone 2 geometry, current or desired blood flow rate, blood viscosity and current or desired blood flow rate range. Analysis may include evaluating enlargement of one or more of the areas 3 to determine if treatment is needed for one or more of the zones to achieve desired blood flow.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of addressing vascular stenotic lesions in the vascular system of a patient, the method comprising:
determining the geometry of an occluded zone in a blood vessel;
estimating pressure drop through the occluded zone using output from a pressure drop algorithm; and
determining if the occluded zone needs treatment based on the estimated pressure drop.

2. The method of claim 1 wherein the geometry being determined in three dimensions.

3. The method of claim 2 wherein the degree of stenosis of the occluded zone being determined from the geometry of the occluded zone.

4. The method of claim 2 wherein the geometry of the occluded zone including both length and transverse area of the occluded zone.

5. The method of claim 4 including treating the occluded zone in accordance with the estimated pressure drop.

6. The method of claim 4 including determining the degree of stenosis using intravascular ultrasound.

7. The method of claim 2 wherein the occluded zone including a stenosis set of at least two adjacent occluded areas and the pressure drop estimation including pressure drop across all the occluded areas in the occluded zone.

8. The method of claim 7 wherein the pressure drop across each said occluded area being estimated individually and treatment options including determining if treatment of one or more of the occluded areas is deemed adequate to achieve a desired pressure drop.

9. The method of claim 2 wherein the output being based on considering at least one of blood flow rate, blood viscosity, blood flow rate range, patient blood pressure, ejection fraction, body mass and patient lifestyle.

10. An apparatus for determining a vascular treatment regimen for an occluded blood vessel, said apparatus including:
a data processing apparatus including a processor and a memory, the data processing apparatus being programmed to provide output data representative of pressure drop in a blood vessel based on input data representative of the geometry of a portion of the blood vessel;
data input means operably connected to the data processing apparatus to permit data input into the data processing apparatus; and
data output means operably connected to the data processing apparatus to permit data output from the data processing apparatus.

11. The apparatus of claim 10 wherein the data input means including an intravascular ultrasound device.

12. The apparatus of claim 10 wherein the data output means including a monitor.

13. The apparatus of claim 10 wherein the data processing means including a digital computer.

14. The apparatus of claim 13 wherein the data processing apparatus being programmed with an algorithm operable to process the input data to provide the output data.
